(19) United States
(12) Patent Application Publication
(10) Pub. No.: US 2022/0241531 A1
(43) Pub. Date: Aug. 4, 2022

(54) TRACHEOSTOMY STABILIZER

(71) Applicant: Mayo Foundation for Medical Education and Research, Rochester, MN (US)

(72) Inventors: William D. Freeman, Jacksonville, FL (US); Phillip Pirgousis, Ponte Vedra Beach, FL (US)

(21) Appl. No.: 17/629,510

(22) PCT Filed: Jul. 31, 2020
(86) PCT No.: PCT/US2020/044475

§ 371 Date: Jan. 24, 2022

Related U.S. Application Data
(60) Provisional application No. 62/882,104, filed on Aug. 2, 2019.

(51) Int. Cl. A61M 16/04; A61M 16/08

(52) U.S. Cl. CPC A61M 16/0465; A61M 16/0816

(57) ABSTRACT

This document relates to a stabilizer for use with a tracheostomy tube assembly that includes a base configured to couple to a patient and a connector coupled to the base, where the connector defines a lumen configured to receive a portion of the tracheostomy tube assembly.

### CROSS-REFERENCE TO RELATED APPLICATIONS

This application claims the benefit of U.S. Provisional Application Ser. No. 62/882,104, filed Aug. 2, 2019. The disclosure of the prior application is considered part of (and is incorporated by reference in) the disclosure of this application.

### BACKGROUND

### 1. Technical Field

This document relates to methods and materials for improving stabilization of tubing on a patient. For example, this document relates to methods and devices for stabilization of tracheostomy tubing on a patient.

### 2. Background Information

Tracheotomy, or tracheostomy, is a surgical procedure which consists of making an incision on the anterior aspect of the neck and opening a direct airway through an incision in the trachea. The resulting stoma can serve independently as an airway or as a site for a tracheal tube or tracheostomy tube to be inserted; this tube allows a person to breathe without the use of the nose or mouth. Percutaneous tracheostomy is a common procedure for critically ill patients who fail to wean off the ventilator and in the otolaryngology practice. Immediately after a percutaneous tracheostomy however the most feared complication is tracheostomy dislodgement which can be life-threating event and even lead to death. The methods by which tracheostomy sites are secured is highly variable. Some surgeons place simple sutures, others use white-cotton shoe string like devices to affix around the neck. However, in Neuro/ICU and some ENT patients with tracheostomy, tight constriction around the entire neck may lead to secondary complications in these patients such as poor circulation (venous or arterial, or raised intracranial pressure).

In a review in the Journal of American College of Surgery of 3,162 tracheostomies in the ICU, early major complication rates included 3 airway losses, 1 bleeding event and 3 procedure-related deaths. However, moderate and lesser or undocumented tracheal injuries or ‘near misses’ of tracheostomy or patient injury occur that is harder to quantify due to motion of the tracheostomy site.

### SUMMARY

This document describes methods and materials for improving stabilization of tubing on a patient. For example, this document describes methods and devices for stabilization of tracheostomy tubing on a patient.

In one aspect, this disclosure is directed to a stabilizer for use with a tracheostomy tube assembly. The stabilizer includes a base configured to couple to a patient and a connector coupled to the base, where the connector defines a lumen configured to receive a portion of the tracheostomy tube assembly.

In some cases, the base can include an intermediate layer configured to couple to the patient. In some cases, the intermediate layer can include an adhesive. In some cases, the stabilizer can further include a removable backing coupled to the adhesive. In some cases, the intermediate layer can include a foam. In some cases, the base can be substantially rigid. In some cases, the base can be flexible. In some cases, the lumen can be substantially smooth. In some cases, an interior of the connector defining the lumen can include a foam. In some cases, the connector can be removably coupled to the base. In some cases, the connector can be rotatably coupled to the base. In some cases, the connector can include an upper portion and a lower portion. In some cases, the upper portion can be configured to move relative to the lower portion between an open position and a closed position. In some cases, the upper portion can be coupled to the lower portion via a hinge. In some cases, the stabilizer can include a securing mechanism that can be configured to maintain the upper portion in the closed position. In some cases, the securing mechanism can be a latch. In some cases, the lower portion can include a groove and the upper portion can include a protrusion configured to engage with the groove to maintain the upper portion in the closed position. In some cases, the stabilizer can include a release tab coupled to the upper portion, where the release tab can be configured to receive a force to move the upper portion from the closed position to the open position. In some cases, the stabilizer further comprises an angle adjuster mechanism configured to adjust the angle of the connector with respect to the base. In some cases, the angle is between 0 degrees to 90 degrees.

In another aspect, this disclosure is directed to a method of stabilizing a tracheostomy tube assembly. The method includes inserting a portion of the tracheostomy tube assembly into a lumen defined by a connector of a stabilizer and coupling a base of the stabilizer to a patient, where the base is coupled to the connector.

In some cases, coupling the base of the stabilizer to the patient can include pressing an adhesive layer of the base onto skin of the patient. In some cases, coupling the base of the stabilizer to the patient can include coupling the base to a shoulder region of the patient. In some cases, coupling the base of the stabilizer to the patient can include coupling the base to a neck region of the patient. In some cases, inserting the portion of the tracheostomy tube assembly into the lumen defined by the connector of the stabilizer can include moving the connector between an open position and a closed position. In some cases, inserting the portion of the tracheostomy tube assembly into the lumen defined by the connector of the stabilizer can further include engaging a securing mechanism to maintain the connector in the closed position. In some cases, the method can include removing the stabilizer after about 1 week to about 3 weeks. In some cases, the method further comprises adjusting an angle of the connector with respect to the base. In some cases, the angle is between 0 degrees to 90 degrees.

In some cases, the method can include inserting a second portion of the tracheostomy tube assembly into a second lumen defined by a second connector of a second stabilizer and coupling a second base of the stabilizer to the patient, where the second base is coupled to the second connector. In some cases, the stabilizer and the second stabilizer can be located on different regions of the patient. In some cases, the stabilizer can be located on a shoulder region of the patient and the second stabilizer can be located on a neck region of the patient.

In another aspect, this disclosure is directed to a system for use with a tracheostomy tube assembly. The system includes a first stabilizer and a second stabilizer. The first stabilizer includes a first base configured to couple to a patient and a first connector coupled to the first base, where the first connector defines a first lumen configured to receive a first portion of the tracheostomy tube assembly. The second stabilizer includes a second base configured to couple to the patient and a second connector coupled to the second base, where the second connector defines a second lumen configured to receive a second portion of the tracheostomy tube assembly.

In some cases, the first lumen can have a first diameter and the second lumen can have a second diameter. In some cases, the first diameter and the second diameter can be different.

Particular embodiments of the subject matter described in this document can be implemented to realize one or more of the following advantages.

First, the stabilizer can reduce injury at a tracheostomy site, which can be injured by motion or torsion imposed which can cause tissue trauma from “pulls and tugs” on the tracheostomy in different directions in 360 degrees. The stabilizer can reduce injury by minimizing motion-related trauma at the surgical site by fixating these tugs/pulls only on the shoulder region. Additionally, the use of multiple stabilizers can further reduce trauma at the insertion site of the tracheostomy. For example, one stabilizer may be located at the shoulder region and a second stabilizer may be located near the neck, closer to the insertion site. The stabilizer at the shoulder region can be a first line of prevention for tugs and pulls at the insertion site, and the second stabilizer at the neck region can be a second line of prevention and pulls at the insertion site. Commonly, patients are turned every two hours in the ICU to prevent bed bottom skin ulcers, but there are additional risks to tracheostomy site movement include transport to CT scan (Neuro) and during mobilization in bed with physical therapy. The systems and methods for the stabilizer provided herein can reduce these risks. The stabilizer could be a single application stabilizer. The stabilizer could be used immediately after the operating room and beside intensive care unit percutaneous tracheostomy. Such use can reduce major complication rates of early tracheostomy dislodgement.

Second, the stabilizer is simple to use. Therefore, the stabilizer can greatly reduce the time requirements for users. For example, this comes up every week and the use of the stabilizer described herein would give back physician time (estimated application time <1 min) versus 15 minutes needed for acquiring and educating various team members, ARNP/PA's, Nursing, respiratory therapists, etc. Third, the stabilizer could be replaced or cleaned if soiled.

Unless otherwise defined, all technical and scientific terms used herein have the same meaning as commonly understood by one of ordinary skill in the art to which this disclosure pertains. Although methods and materials similar or equivalent to those described herein can be used to practice the various embodiments, suitable methods and materials are described herein. All publications, patent applications, patents, and other references mentioned herein are incorporated by reference in their entirety. In case of conflict, the present specification, including definitions, will control. In addition, the materials, methods and examples are illustrative only and not intended to be limiting.

The details of one or more embodiments of the disclosure are set forth in the accompanying drawings and the description below. Other features, objects, and advantages of the methods and devices described will be apparent from the description, drawings, and from the claims.

### DESCRIPTION OF DRAWINGS

FIG. 1 is a schematic diagram of a stabilizer positioned on a shoulder of a patient with a tracheostomy tube assembly, in accordance with some embodiments provided herein.

FIG. 2 is a close up schematic diagram of a stabilizer positioned on a tracheostomy tube assembly, in accordance with some embodiments provided herein.

FIG. 3 is a schematic diagram of multiple stabilizers positioned on a tracheostomy tube assembly, in accordance with some embodiments provided herein.

FIG. 4 is a perspective view of a stabilizer, in accordance with some embodiments provided herein.

FIG. 5 is a side view of the stabilizer of FIG. 4, in accordance with some embodiments provided herein.

FIG. 6 is a perspective view of the stabilizer of FIG. 4 in an open position, in accordance with some embodiments provided herein.

FIG. 7 is a side view of an example stabilizer positioned at an angle with respect to the base, in accordance with some embodiments provided herein.

Like reference numbers represent corresponding parts throughout.

### DETAILED DESCRIPTION

This document describes methods and materials for improving stabilization of tubing on a patient. For example, this document describes methods and devices for stabilization of tracheostomy tubing on a patient.

Tracheotomy, or tracheostomy, is a surgical procedure which consists of making an incision on the anterior aspect of the neck and opening a direct airway through an incision in the trachea. The resulting stoma can serve independently as an airway or as a site for a tracheal tube or tracheostomy tube to be inserted; this tube allows a person to breathe without the use of the nose or mouth.

The stabilizer provided herein can reduce injury at a tracheostomy site by providing stabilization of a tracheostomy tube assembly to limit movement of the tracheostomy tubing at the incision site. The stabilizer can be simple to use and can be replaced or cleaned if soiled, saving time and effort of users.

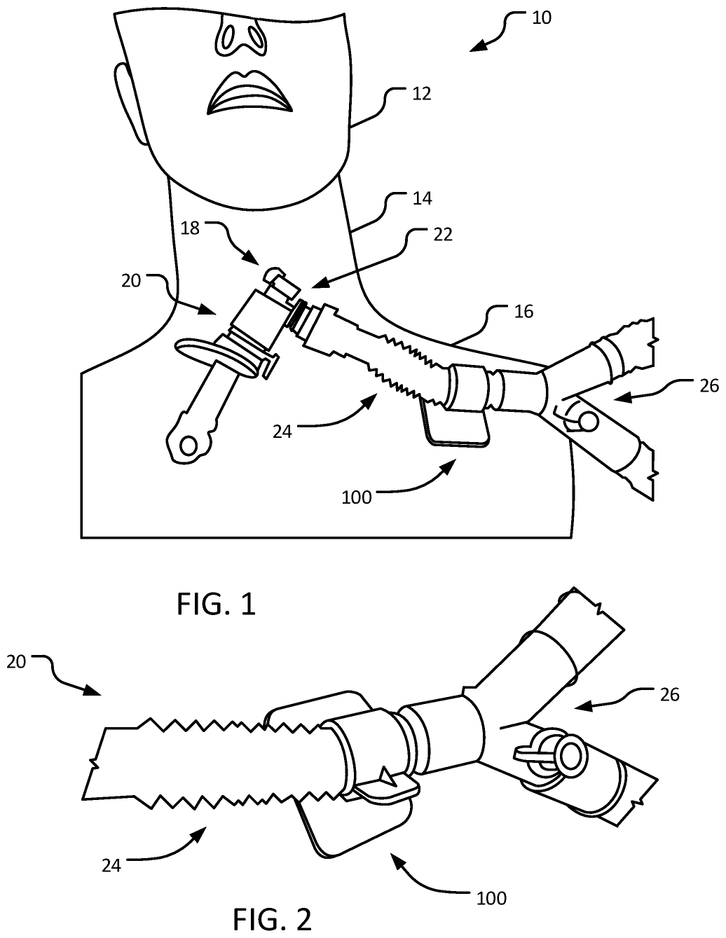

Referring to FIGS. 1 and 2, once an opening 18 has been created in a neck 14 of a patient 10, a tracheostomy tube 22 of a tracheostomy tube assembly 20 can be positioned in opening 18.

Tracheostomy tube assembly 20 can include extendable tubing 24 located along various portions of tracheostomy tube assembly 20. For example, extendable tubing 24 can have an accordion shape that allows stretching of extendable tubing 24 to modify a length of extendable tubing 24. In some cases, tracheostomy tube assembly 20 can include a tubing junction 26 where tubing branches off to various locations.

A stabilizer 100 can be connected to tracheostomy tube assembly 20 to aid in securing tracheostomy tube assembly 20 to patient 10. For example, movement of a head 12 of patient 10 may cause movement of neck 14 and therefore movement of tracheostomy tube assembly 20, which can be dangerous. In some cases, stabilizer 100 can be used immediately post-operatively. In some cases, stabilizer 100 can be used for a healing period. In some cases, the healing period is about 1 to about 3 weeks. In some cases, stabilizer 100 can be used until the tissue surrounding opening 18 of neck 14 is healed. In some cases, stabilizer 100 can be removed and replaced throughout the healing period. In some cases, stabilizer 100 can be removed and sanitized throughout the healing period.

In some cases, stabilizer 100 can be positioned near a shoulder 16 of the patient 10. In some cases, stabilizer 100 can be positioned, at, above, or below a collar bone of patient 10. In some cases, stabilizer 100 can be positioned proximal shoulder 16. In some cases, stabilizer 100 can be positioned proximal neck 14. While stabilizer 100 is shown connected to extendable tubing 24, stabilizer 100 can be configured to couple with any portion of tracheostomy tube assembly 20.

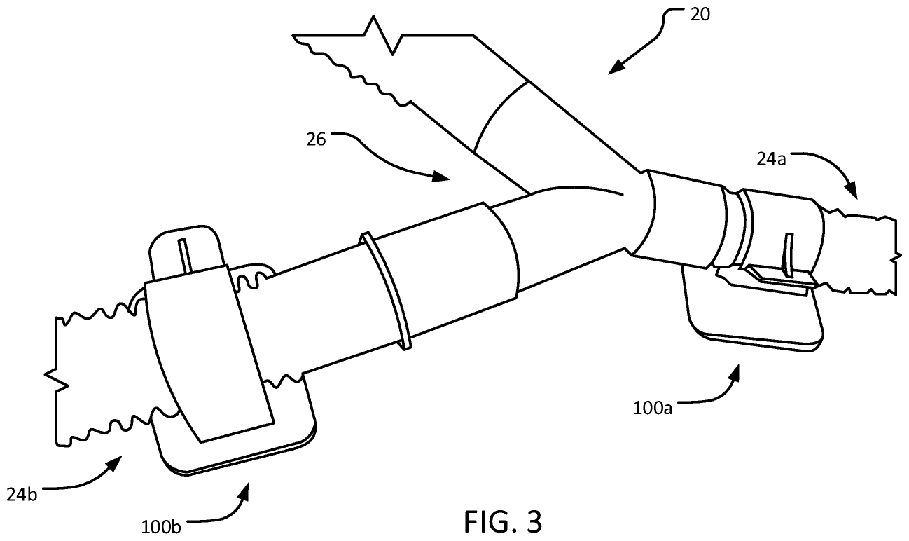

Referring to FIG. 3, multiple stabilizers 100 can be positioned on the tracheostomy tube assembly 20. For example, a first stabilizer 100a can be positioned at a first location along the tracheostomy tube assembly 20 and a second stabilizer 100b can be positioned at a second location along the tracheostomy tube assembly 20. In some cases, first stabilizer 100a can be positioned before tubing junction 26 along a first portion of extendable tubing 24a, while second stabilizer 100b can be positioned after tubing junction 26 along a second portion of extendable tubing 24b.

In some cases, first stabilizer 100a or second stabilizer 100b can be positioned near neck 14 of patient 10 and the other of first stabilizer 100a or second stabilizer 100b can be positioned near shoulder 16 of patient 10. In some cases, a first point of attachment (e.g., via first stabilizer 100a) can provide smooth movement of tubing during movement of head 12 and/or neck 14. In some cases, a second point of attachment (e.g., via second stabilizer 100b) can provide additional security for more drastic movement of patient 10. In some cases, the second point of attachment (e.g., near shoulder 16 of patient 10) can cause movement of tracheostomy tube assembly 20 to pull on shoulder 16 and/or stabilizer 100 located at shoulder 16 instead of tracheostomy tube 22 inside opening 18 of neck 14.

In some cases, first stabilizer 100a and second stabilizer 100b can have different calibers. For example, extendable tubing 24a and 24b can have a different diameter, therefore first stabilizer 100a and second stabilizer 100b can have different diameters to allow for coupling with extendable tubing 24a and 24b. In some cases, extendable tubing 24 proximal neck 14 of patient 10 can have a smaller diameter than extendable tubing 24 distal neck 14 (e.g., proximal shoulder 16) of patient 10.

In some cases, first stabilizer 100a and second stabilizer 100b can come packaged together. In some cases, first stabilizer 100a and second stabilizer 100b can include unique markings to differentiate between the first stabilizer 100a and the second stabilizer 100b. For example, the marking can be a character, a letter, a number, a symbol, a size, a color, or other indicator to differentiate between first stabilizer 100a and second stabilizer 100b.

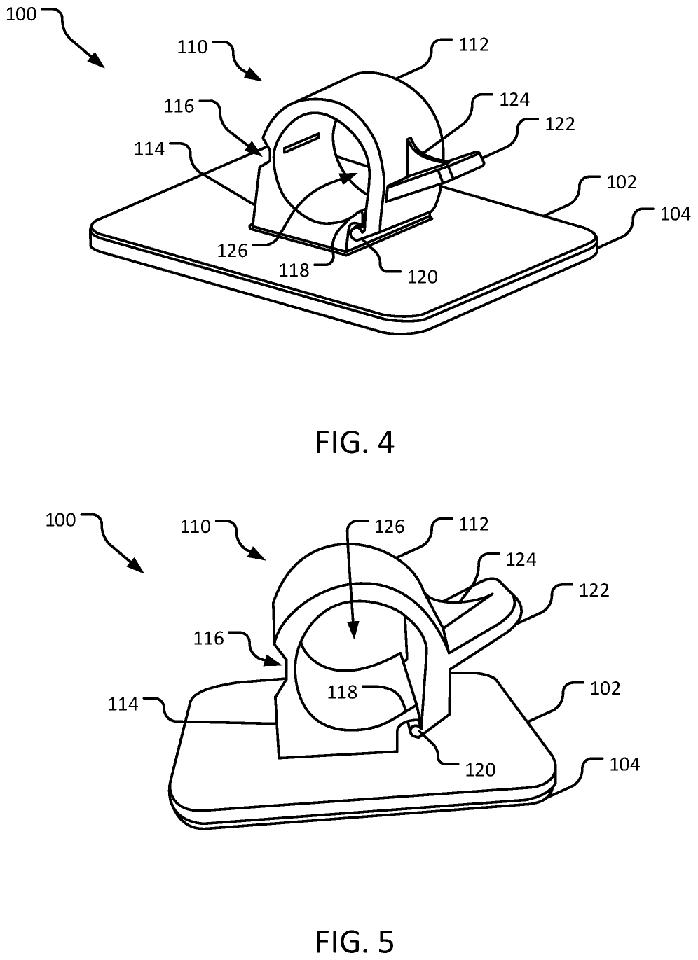

Referring to FIGS. 4-6, stabilizer 100 can include a base 102 and a connector 110. Stabilizer 100 can be configured to engage with tracheostomy tube assembly 20 to allow movement of tubing (e.g., extendable tubing 24) through stabilizer 100, such as longitudinal movement, but limit, reduced, or inhibit movement of portions of tracheostomy tube assembly 20 (e.g., tubing) away from patient 10 (e.g., away from skin of patient 10). In some cases, stabilizer 100 can be made of plastic, metal, or other material that can be sterilized. In some cases, stabilizer 100 can be 3D printed.

Base 102 can be configured to provide support for connector 110 and couple to patient 10. In some cases, base 102 can be substantially rigid. In some cases, base 102 can be flexible, such that base 102 can conform to contours on patient 10. In some cases, base 102 can include an adhesive backing to couple stabilizer 100 to patient 10. In some cases, base 102 can be substantially flat. In some cases, base 102 can be curved to fit contours of a standard patient.

In some cases, base 102 can include an intermediate layer 104 on an underside of base 102, such that intermediate layer 104 is configured to engage with the patient 10 (e.g., a skin of patient 10). In some cases, intermediate layer 104 can be a foam, such that intermediate layer 104 can conform to patient 10. In some cases, intermediate layer 104 can include an adhesive layer. In some cases, the adhesive layer can include a removable backing.

Connector 110 can include an interior surface defining a lumen 126 configured to receive a portion of tracheostomy tube assembly 20. For example, lumen 126 can be substantially cylindrical. In some cases, a diameter of lumen 126 can be substantially similar to a diameter of extendable tubing 24.

In some cases, interior surface of connector 110 can be smooth. In some cases, interior surface of connector 110 can be a foam that can act as a microstabilizer.

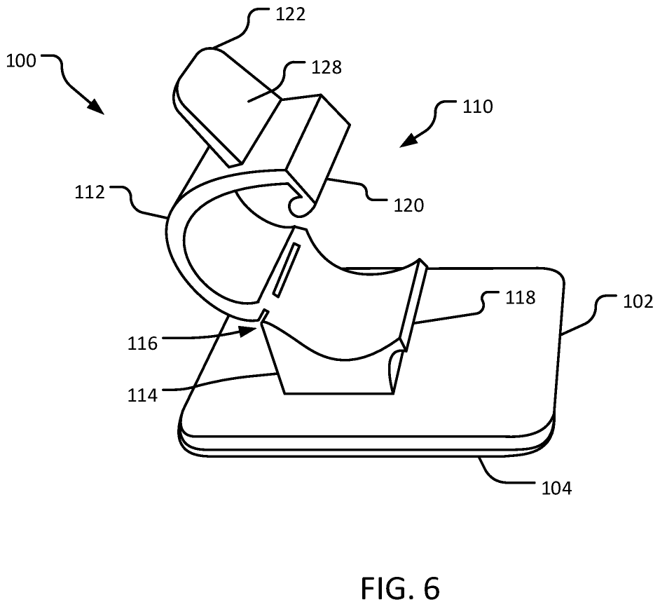

In some cases, connector 110 can include an upper portion 112 and a lower portion 114 that are coupled together. In some cases, upper portion 112 and lower portion 114 can be coupled together via a hinge 116. In some cases, hinge 116 can be plastic. In some cases, hinge 116 can be a flexible portion of connector 110 extending between upper portion 112 and lower portion 114. In some case, hinge 116 can be made of a different material than connector 110. Hinge 116 can be configured to move upper portion 112 between a closed position (as shown in FIGS. 4 and 5) and an open position (as shown in FIG. 6). When in the open position, connector 110 can receive a portion of tracheostomy tube assembly 20. When in the closed position, connector 110 can allow longitudinal movement of portions of tracheostomy tube assembly 20, while limiting, reducing, or inhibiting movement of at least portions of tracheostomy tube assembly 20 away from patient 10.

Lower portion 114 can be coupled to base 102. In some cases, lower portion 112 can be rigid. In some cases, lower portion 114 can be removably coupled to base 102. In some cases, lower portion 114 can be rotatably coupled to base 102. For example, lower portion 114 may be able to rotate about 45 degrees from a longitudinal direction of lumen 126 in two directions. In some cases, lower portion 114 can include a securing mechanism 118 opposite hinge 116. Securing mechanism 118 can define a groove on an underside that can be configured to engage with a portion of upper portion 112 to maintain connector 110 in a closed position.

In some cases, lower portion 114 and upper portion 112 can engage with one another to maintain a closed position via a latch, a snap-fit, a hook and loop connector, a pin and slot connection, an interference fit, or other means for maintaining a closed position Upper portion 112 can move relative to lower portion 114, such that connector 110 can move between an open position and a closed position. In some cases, upper portion 112 can include a securing mechanism 120 that can be configured to engage with the securing mechanism 118 of lower portion 114. In some cases, securing mechanism 120 can be a protrusion that latches into the groove of securing mechanism 118. In some cases, the securing mechanism 120 can be rounded. For example, the rounded profile of securing mechanism 120 can aid with smooth engagement and disengagement with securing mechanism 118.

In some cases, upper portion 112 can include a release tab 122 that is secured to upper portion 112 via support 124. Release tab 122 can provide a surface for a user to engage with to release securing mechanism 120 from securing mechanism 118. In some cases, force can be applied to release tab 122 substantially perpendicular to a front surface 128 of release tab 122 to cause disengagement of securing mechanism 120 from securing mechanism 118. In some cases, upper portion 112, or portions of upper portion 112, can be slightly flexible to allow securing mechanism 120 to engage and disengage with securing mechanism 118 without damage.

A stabilizer may be substantially similar in construction and function in several aspects to the stabilizer 100 discussed above but can include an alternative connector 710. In some embodiments, the alternative connector may have two configurations such that the connector can be either positioned parallel to the base or at an angle with the base. For example, the connector may be positioned at an angle between 0 degrees and 90 degrees with respect to the base. Such adjustable angular configurations can allow a user (e.g., a clinician) to manipulate the angle of the connector in order to provide some slack to the tubing being secured; thereby, reducing the probability of dislodgement and/or painful movement of the tracheostomy tubing at the insertion site.

Furthermore, angular adjustment of the stabilizer and tracheostomy tube assembly can be important to individualize and adjust for each patient. Each patient has a different tracheostomy angle (e.g., the entry angle of the tracheostomy tube into a trachea of the patient) relative to their chest and a different dynamic motion of their head and neck relative to their chest. Moreover, each patient can have a different height and/or can be of a different size. Thus, the tracheostomy tube assembly stabilizers of the disclosure can include features (e.g., an angle adjuster mechanism) that allow a user to advantageously adjust and individualize the angle at which the connector, base, or both is positioned relative to the plane of the tracheostomy, chest, head, and/or neck of the patient in order to provide some slack to the tracheostomy tube secured to the patient.

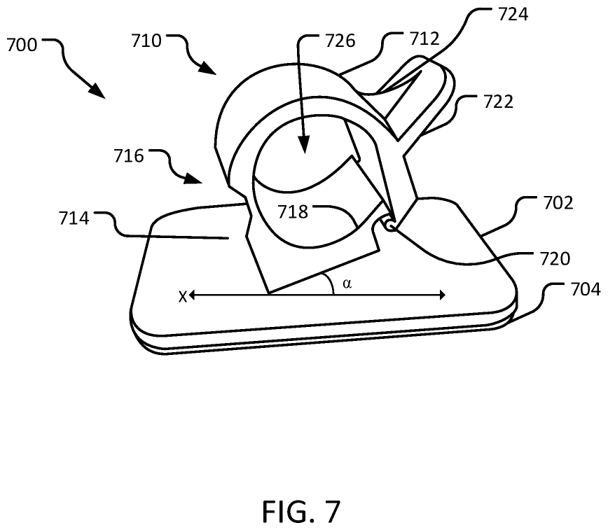

Referring to FIG. 7, stabilizer 700 can include a base 702 and a connector 710. Connector 710 can include an angle adjuster mechanism that is configured to position the connector 710 at an angle α with respect to the base 702. That is, angle α is defined as the angle of the connector 710 with respect to a horizontal axis X of the base 702. The angle adjuster mechanism can allow the connector 710 to be positioned at angle increments of 15 degrees with respect to the base 702. In some embodiments, angle α is 0 degrees. In some embodiments, angle α is 15 degrees. In some embodiments, angle α is 45 degrees. In some embodiments, angle α is 90 degrees. In some embodiments, angle α ranges from about 0 degrees to about 90 degrees or more. In some embodiments, angle α ranges from about 0 to 10, 10 to 15 degrees, 15 to 30 degrees, 30 to 45 degrees, 45 to 60 degrees, 60 to 75 degrees, 75 to 90 degrees, or more. In some embodiments, the angle adjuster mechanism is configured to reversibly adjust the angle α of the connector 710 with respect to the base 702.

In some embodiments, the base 702 is adjustable. For example, base 702 can be adjusted such that the angle of the base 702 with respect to the intermediate layer 704 can be varied. In some embodiments, connector 710 includes an angle adjuster mechanism that is configured to adjust the angle of the base 702 with respect to the intermediate layer 704. The angle adjuster mechanism can allow the base 702 to be positioned at angle increments of 15 degrees with respect to the intermediate layer 704. In some embodiments, the angle of the base 702 with respect to the intermediate layer 704 is 0 degrees. In some embodiments, the angle of the base 702 with respect to the intermediate layer 704 is 15 degrees. In some embodiments, the angle of the base 702 with respect to the intermediate layer 704 is 45 degrees. In some embodiments, the angle of the base 702 with respect to the intermediate layer 704 is 90 degrees. In some embodiments, the angle of the base 702 with respect to the intermediate layer 704 ranges from about 0 degrees to about 90 degrees or more. In some embodiments, the angle of the base 702 with respect to the intermediate layer 704 ranges from about 0 to 10 degrees, 10 to 15 degrees, 15 to 30 degrees, 30 to 45 degrees, 45 to 60 degrees, 60 to 75 degrees, 75 to 90 degrees, or more. In some embodiments, the angle adjuster mechanism is configured to reversibly adjust the angle of the base 702 with respect to the intermediate layer 704.

Non-limiting examples of angle adjuster mechanisms include a gear and lock mechanism and a ratchet mechanism. In some embodiments, the angle adjuster mechanism adjusts the connector 710 or the base 702 by varying angles. In some embodiments, the angle adjuster mechanism can include a pawl that engages the teeth of a gear every time the angle is adjusted. In some embodiments, the user can lift or depress the connector 710 with respect to the base 702 in order to adjust the angle α. In some embodiments, the user can lift or depress the base 702 with respect to the intermediate layer 704 in order to adjust the angle defined therebetween. In some cases, the user may hear a “click” sound for every angle increment or angle decrement that the angle adjuster mechanism generates via user input.

In an aspect, the present disclosure is directed to systems including two or more stabilizers. In some embodiments, the two or more stabilizers can include angle adjustment mechanisms. For example, a user (e.g., a clinician) can reversibly attach a first stabilizer 710 and a second stabilizer 710 to the skin surface of a patient in order. In some embodiments, the system of two or more stabilizers can allow increased slack in the tracheostomy tubing, as compared to using one or more stabilizers 110 and/or as compared to using one stabilizer 710. Furthermore, in some embodiments, the system of two or more stabilizers can permit greater range of motion of the patient without causing dislodgement and/or painful movement (e.g., “pulls and tugs”) of the tracheostomy tubing at the insertion site.

Similar to connector 110, connector 710 can include an interior surface defining a lumen 726 configured to receive a portion of tracheostomy tube assembly 20. In some cases, interior surface of connector 710 can be smooth. In some cases, interior surface of connector 710 can be a foam that can act as a microstabilizer. In some cases, connector 710 can include an upper portion 712 and a lower portion 714 that are coupled together. In some cases, upper portion 712 and lower portion 714 can be coupled together via a hinge 716. Similar to the components of connector 110, upper portion 712 and lower portion 714 can operate in the same manner.

Lower portion 714 can be coupled to base 702. In some cases, lower portion 712 can be rigid. In some cases, lower portion 714 can be removably coupled to base 702. In some cases, lower portion 714 can be rotatably coupled to base 702. For example, lower portion 714 may be able to rotate about 45 degrees from a longitudinal direction of lumen 726 in two directions. In some cases, lower portion 714 can include a securing mechanism 718 opposite hinge 716. Securing mechanism 718 can define a groove on an underside that can be configured to engage with a portion of upper portion 712 to maintain connector 710 in a closed position.

Upper portion 712 can move relative to lower portion 714, such that connector 710 can move between an open position and a closed position. In some cases, upper portion 712 can include a securing mechanism 720 that can be configured to engage with the securing mechanism 718 of lower portion 714. In some cases, securing mechanism 720 can be a protrusion that latches into the groove of securing mechanism 718. In some cases, the securing mechanism 720 can be rounded. For example, the rounded profile of securing mechanism 720 can aid with smooth engagement and disengagement with securing mechanism 718.

In some cases, upper portion 712 can include a release tab 722 that is secured to upper portion 712 via support 724. Release tab 722 can provide a surface for a user to engage with to release securing mechanism 720 from securing mechanism 718. In some cases, force can be applied to release tab 722 substantially perpendicular to a front surface of release tab 722 to cause disengagement of securing mechanism 720 from securing mechanism 718. In some cases, upper portion 712, or portions of upper portion 712, can be slightly flexible to allow securing mechanism 720 to engage and disengage with securing mechanism 718 without damage.

While this specification contains many specific implementation details, these should not be construed as limitations on the scope of any method or device or of what may be claimed, but rather as descriptions of features that may be specific to particular embodiments of particular methods and devices. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the process depicted in the accompanying figures do not necessarily require the particular order shown, or sequential order, to achieve desirable results. In certain implementations, multitasking and parallel processing may be advantageous.

1.-35. (canceled)

36. A stabilizer for use with a tracheostomy tube assembly, comprising:
a base configured to couple to a patient; and
a connector coupled to the base, the comprising an upper portion and a lower portion, wherein the connector defines a lumen configured to receive a portion of the tracheostomy tube assembly.

37. The stabilizer of claim 36, wherein the base comprises an intermediate layer configured to coupled to the patient, wherein the intermediate layer comprises an adhesive and a removable backing coupled to the adhesive.

38. The stabilizer of claim 37, wherein the intermediate layer comprises a foam.

39. The stabilizer of claim 36, wherein the base is substantially rigid or flexible.

40. The stabilizer of claim 36, wherein the lumen is substantially smooth.

41. The stabilizer of claim 36, wherein an interior of the connector defining the lumen comprises a foam.

42. The stabilizer of claim 36, wherein the connector is removably coupled to the base or rotatably coupled to the base.

43. The stabilizer of claim 36, wherein the upper portion is configured to move relative to the lower portion between an open position and a closed position.

44. The stabilizer of claim 43, further comprising a securing mechanism configured to maintain the upper portion in the closed position, wherein the securing mechanism is a latch.

45. The stabilizer of claim 43, wherein the lower portion comprises a groove and the upper portion comprises a protrusion configured to engage with the groove to maintain the upper portion in the closed position.

46. The stabilizer of claim 43, further comprising a release tab coupled to the upper portion, wherein the release tab is configured to receive a force to move the upper portion from the closed position to the open position.

47. The stabilizer of claim 36, wherein the upper portion is coupled to the lower portion via a hinge.

48. The stabilizer of claim 36, further comprising an angle adjuster mechanism configured to adjust an angle of the connector with respect to the base.

49. The stabilizer of claim 48, wherein the angle is between 0 degrees to 90 degrees.

50. A method of stabilizing a tracheostomy tube assembly, comprising:
moving a connector of a stabilizer into an open position;
inserting a portion of the tracheostomy tube assembly into a lumen defined by the connector of the stabilizer;
coupling a base of the stabilizer to a patient, wherein the base is coupled to the connector; and
moving the connector of the stabilizer into a closed position,
wherein coupling the base of the stabilizer to the patient comprises pressing an adhesive layer of the base onto skin of the patient.

51. The method of claim 50, wherein inserting the portion of the tracheostomy tube assembly into the lumen defined by the connector of the stabilizer further comprises engaging a securing mechanism to maintain the connector in the closed position.

52. The method of claim 50, further comprising removing the stabilizer after about 1 week to about 3 weeks.

53. The method of claim 50, further comprising adjusting an angle of the connector with respect to the base, wherein the angle is between 0 degrees to 90 degrees.

54. The method of claim 50, further comprising:
inserting a second portion of the tracheostomy tube assembly into a second lumen defined by a second connector of a second stabilizer; and
coupling a second base of the stabilizer to the patient, wherein the second base is coupled to the second connector,
wherein the stabilizer and the second stabilizer are located on different regions of the patient.

55. A system for use with a tracheostomy tube assembly, comprising:
a first stabilizer, comprising:
a first base configured to couple to a patient; and
a first connector coupled to the first base, wherein the first connector defines a first lumen configured to receive a first portion of the tracheostomy tube assembly; and
a second stabilizer, comprising:
a second base configured to be coupled to the patient; and
a second connector coupled to the second base, wherein the second connector defines a second lumen configured to receive a second portion of the tracheostomy tube assembly,
wherein the first lumen has a first diameter that is different than a second diameter of the second lumen.
